(19) United States
(12) Patent Application Publication
(10) Pub. No.: US 2012/0165884 A1
(43) Pub. Date: Jun. 28, 2012

(54) FLUID ACCUMULATION MONITORING DEVICES, SYSTEMS AND METHODS

(76) Inventors: Cecilia Qin Xi, San Jose, CA (US); Stuart Rosenberg, Castaic, CA (US); Jong Gill, Valencia, CA (US); Yelena Nabutovsky, Sunnyvale, CA (US); William Hsu, Thousand Oaks, CA (US); Brian Jeffrey Wenzel, San Jose, CA (US)

(21) Appl. No.: 12/975,794

(22) Filed: Dec. 22, 2010

(51) Int. Cl. A61B 5/053; A61N 1/39; A61N 1/362

(52) U.S. Cl. 607/4; 600/547

(57) ABSTRACT

Provided herein are implantable systems, and methods for use therewith, for monitoring a patient's fluid accumulation level. A thoracic impedance signal for the patient is obtained. Based on the thoracic impedance signal, a duration metric indicative of a duration of drop of the thoracic impedance signal, a magnitude metric indicative of a magnitude of drop of the thoracic impedance signal, and a rate metric indicative of a rate of drop of the thoracic impedance signal is determined. The patient's fluid accumulation level is monitored based on the duration metric, the magnitude metric and the rate metric.

### FIELD OF THE INVENTION

Embodiments of the present invention relate to devices, systems and methods for monitoring fluid accumulation in a patient's thoracic cavity.

### BACKGROUND OF THE INVENTION

Heart failure (HF) is often a result of abnormal fluid accumulation in the thoracic cavity, which is also referred to as pulmonary congestion or pulmonary edema. In recent years, impedance based pulmonary congestion detection has been used in heart failure monitoring. Using very low electrical pulses that travel across the thoracic cavity (the chest area encompassing the lungs and heart), an implantable device can measure the level of resistance to the electrical pulses, which indicates the level of fluid in the chest. Determining the level of fluid in the chest by measuring the level of resistance to the electrical pulses is based on the concept that pulmonary fluid retention leads to decreased electrical impedance in cardiac tissue. A record of thoracic impedance can thus serve as a way to monitor HF status, and also to predict HF exacerbations so that timely intervention can be performed and thereby hospitalization can be prevented.

A known technique used to detect abnormal fluid accumulation in the thoracic cavity compares a short-term average thoracic impedance to a long-term average thoracic impedance, and when the long-term average is lower than the short-term average, a fluid index starts to accumulate. When the cumulative fluid index crosses a predetermined threshold, the patient is notified of a possible abnormal fluid level and is instructed to visit a physician or clinician. The physician or clinician then determines whether the patient does, in fact, have an abnormal fluid level that requires treatment.

However, the above described cumulative fluid index does not provide an accurate way to discriminate between safe and unsafe levels of fluid accumulation since a detection of pulmonary congestion is triggered when the fluid index crosses the threshold, regardless of whether the fluid accumulation has any correlation to pulmonary congestion. Further, the above described cumulative fluid index does not provide a way to differentiate between severities and/or causes of congestion. Additionally, adjusting the threshold for the fluid index to better discriminate between safe and unsafe levels of fluid accumulation is not intuitive since there is no clear relationship between the threshold and the fluid index. The above described technique has also resulted in a high false positive detection rate of abnormal fluid accumulation.

Accordingly, it would be beneficial to provide improved techniques for detecting abnormal fluid accumulation levels that have the ability to differentiate levels of severity and/or causes of the congestion.

### SUMMARY

Certain embodiments of the present invention relate to implantable systems and methods for use therewith, for monitoring fluid accumulation in a patient's thoracic cavity.

In an embodiment, one or more electrodes implanted within, on and/or around the patient's thoracic cavity are used to obtain a thoracic impedance signal. Based on the thoracic impedance signal, there is a determination of a duration metric indicative of a duration of drop of the thoracic impedance signal, a magnitude metric indicative of a magnitude of drop of the thoracic impedance signal, and a rate metric indicative of a rate of drop of the thoracic impedance signal.

In certain embodiments, a patient's fluid accumulation level can be monitored based on the duration metric, the magnitude metric and the rate metric. For example, the patient's fluid accumulation level can be monitored by comparing the duration metric to a duration threshold, comparing the magnitude metric to a magnitude threshold, comparing the rate metric to a rate threshold, and then monitoring for an abnormal fluid accumulation level based on the results of the comparisons. In accordance with an embodiment, an alert, and/or therapy and/or adjusting therapy is triggered based on detecting an abnormal fluid accumulation level.

Additional and alternative embodiments, features and advantages of the invention will appear from the following description in which the preferred embodiments have been set forth in detail, in conjunction with the accompanying drawings and claims.

### BRIEF DESCRIPTION OF THE FIGURES

FIG. 1A illustrates exemplary impedance and fluid index graphs that are used to explain a known technique for monitoring fluid levels, where an abnormal fluid level was correctly detected.

FIG. 1B illustrates exemplary impedance and fluid index graphs, where an abnormal fluid level was falsely detected using the same known technique discussed with reference to FIG. 1A.

FIG. 2 is a high level flow diagram that is used to explain various embodiments of the present invention that can be used for monitoring a fluid accumulation level.

FIG. 3A is a high level flow diagram that is used to explain embodiments of the present invention that can be used for determining a duration metric indicative of a duration of drop of a thoracic impedance signal.

FIG. 3B illustrates an exemplary thoracic impedance signal, and illustrates various feature of the thoracic impedance signal that can be used for determining a duration metric indicative of a duration of drop of a thoracic impedance signal, in accordance with the embodiment of the present invention described in FIG. 3A.

FIG. 4 illustrates an exemplary implantable cardiac stimulation device that includes an impedance sensor, which can be used to perform various embodiments of the present invention.

FIG. 5 is a simplified block diagram that illustrates possible components of the implantable device shown in FIG. 4.

### DETAILED DESCRIPTION OF THE INVENTION

The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout. In addition, the first digit of a reference number identifies the drawing in which the reference number first appears.

It would be apparent to one of skill in the art that the present invention, as described below, may be implemented in many different embodiments of hardware, software, firmware, and/or the entities illustrated in the figures. Any actual software, firmware and/or hardware described herein is not limiting of the present invention. Thus, the operation and behavior of the present invention will be described with the understanding that modifications and variations of the embodiments are possible, given the level of detail presented herein.

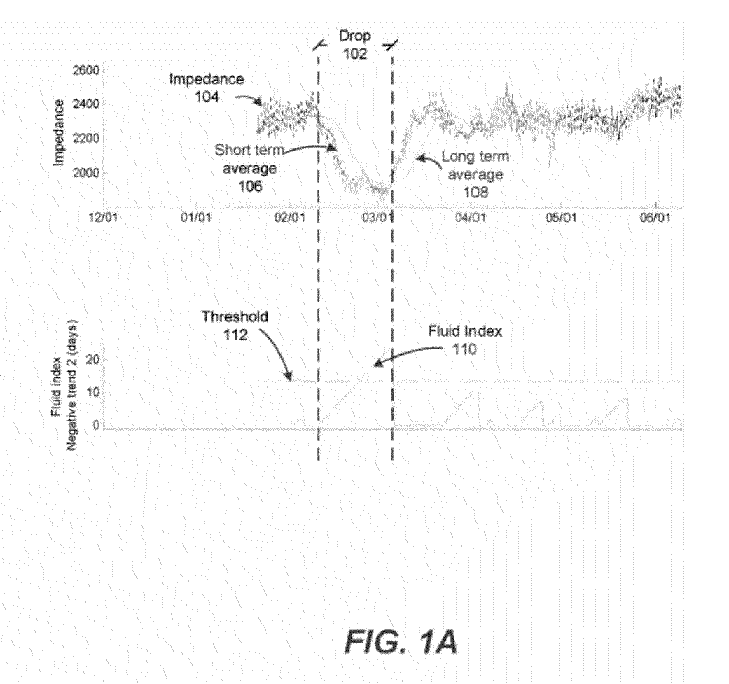

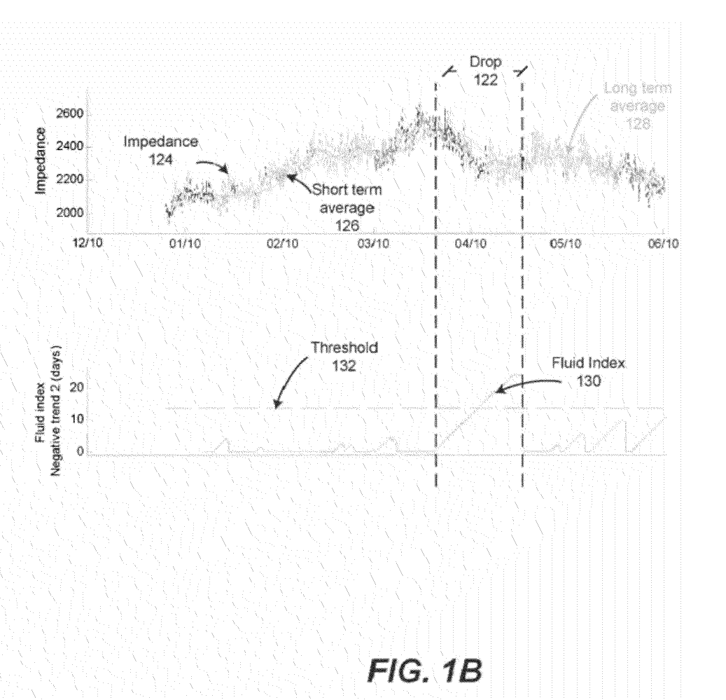

Referring to FIGS. 1A and 1B, the representative graphs and signal waveforms therein are used to explain a known technique for monitoring fluid accumulation levels, where an abnormal fluid accumulation level was correctly detected (in FIG. 1A), and an abnormal fluid accumulation level was falsely detected (in FIG. 1B). In FIGS. 1A and 1B, the upper graphs are of thoracic impedance (measured in ohms) versus time (measured in months). Values represented on the plot include impedance values 104, short-term average impedance values 106, and long-term average impedance values 108. The short-term average impedance values 106 are compared to the long-term average impedance values 108, and when the short-term values 106 are lower than the long-term values 108 (e.g., see drop 102), a fluid index 110 (shown in the lower graph) begins to accumulate. Detection of fluid accumulation is triggered when the fluid index 110 reaches a threshold 112.

During the period of time represented in FIG. 1A, an abnormal fluid accumulation level was properly detected when the fluid index 110 exceeded the threshold 112. Thus, it would have been appropriate for a patient to visit a physician or clinician when informed of the abnormal fluid accumulation level. In contrast, during the period of time represented in FIG. 1B, there was a false detection of an abnormal fluid accumulation level when the fluid index 130 exceeded the threshold 132. This could have resulted in the patient unnecessarily (in terms of cost and time) visiting a physician or clinician, only to be sent home without treatment.

Specific embodiments of the present invention, as will be described below, help to avoid false detections by monitoring fluid accumulation based on multiple metrics, that when taken together, provide for better detection of abnormal fluid accumulation levels. Abnormal fluid accumulation can be due to pulmonary edema, i.e., fluid accumulation in the lungs, and/or fluid accumulation in other organs and/or tissue in the patient's thoracic cavity.

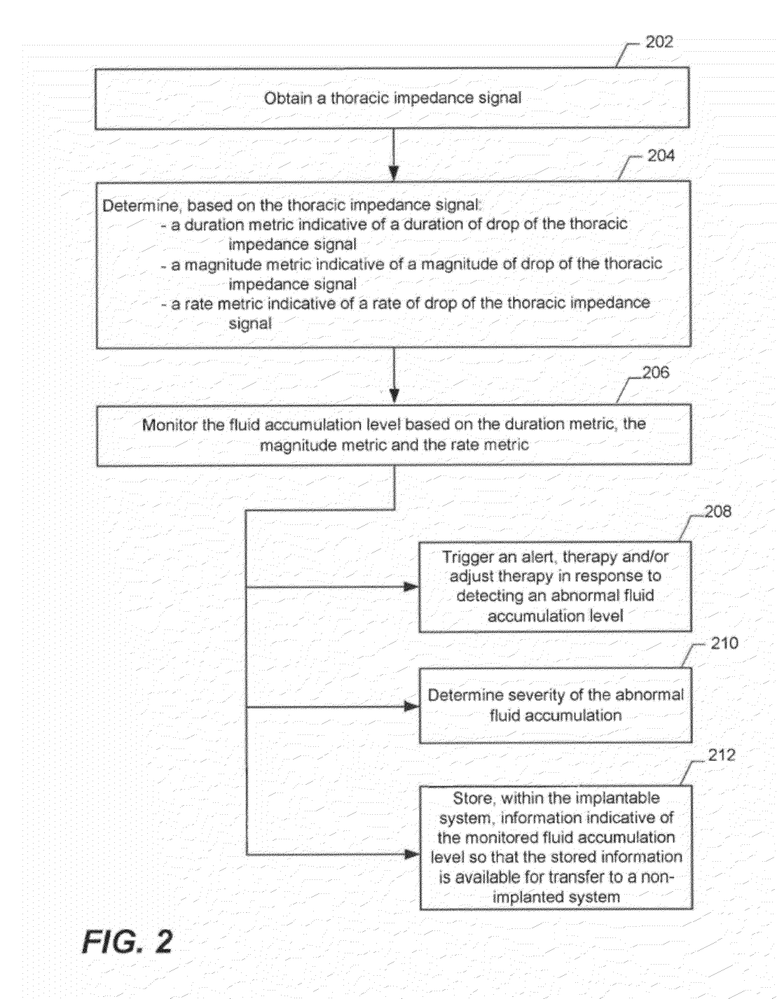

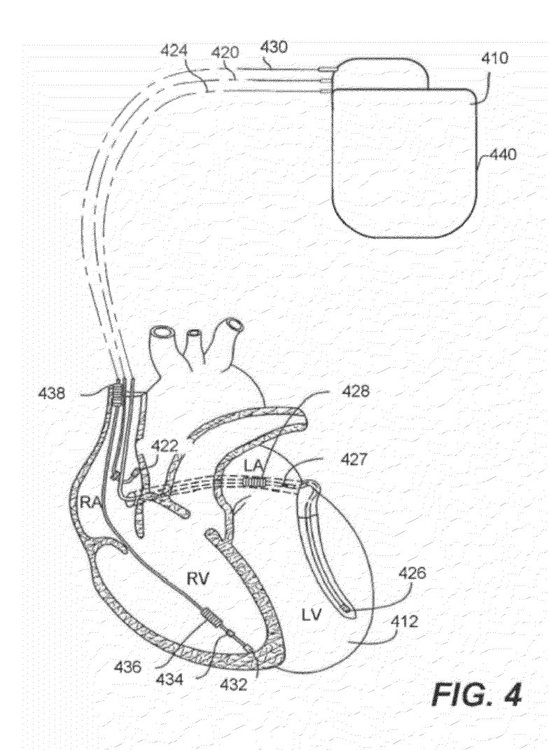

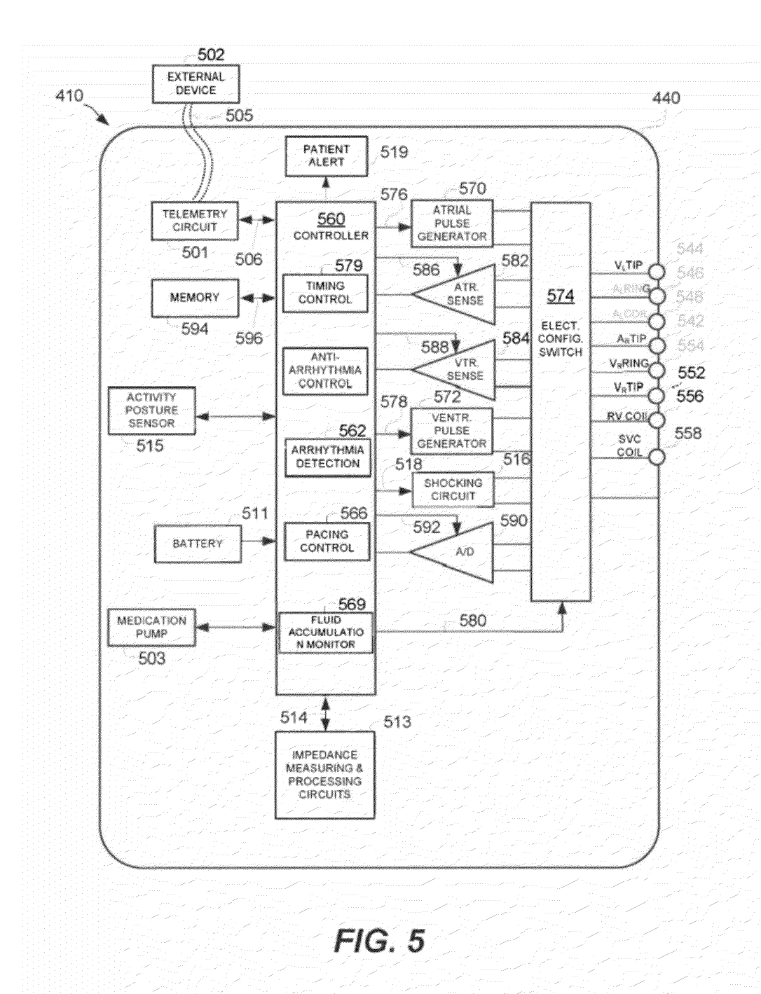

The high level flow diagram of FIG. 2 will now be used to explain various embodiments of the present invention that can be used for monitoring a fluid accumulation level. Such embodiments can be implemented by an implantable system, examples of which are discussed below with reference to FIGS. 4 and 5. In FIG. 2 and the other flow diagrams described herein, the various algorithmic steps are summarized in individual ‘blocks’ Such blocks describe specific actions or decisions that are made or carried out as the algorithm proceeds. Where a microcontroller (or equivalent) is employed, the flow diagram presented herein provides the basis for a ‘control program’ that may be used by such a microcontroller (or equivalent) to effectuate the desired control of the implantable system. Those skilled in the art may readily write such a control program based on the flow diagram and other descriptions presented herein.

Referring to FIG. 2, at step 202, one or more electrodes implanted within, on and/or around the patient's thoracic cavity is/are used to obtain a thoracic impedance signal. Examples of electrodes and circuitry that can be used to obtain a thoracic impedance signal are discussed below with reference to FIGS. 4 and 5. In an embodiment, the thoracic impedance signal obtained at step 202 (and used at step 204 to determine various metrics) can be an impedance signal obtained from more than one impedance vector, combined equally or in a weighted manner. In accordance with an embodiment, the thoracic impedance signal obtained at step 202 can be collected a predetermined number of times per day (e.g., one measurement every two hours), and a short-term average of such measurements can be generated using the collected measurements. A short term average can be generated, e.g., once per day, or each time a new impedance measurement is obtained, but is not limited thereto. For a specific example, each time a new impedance measurement is obtained, the new measurement can be averaged with a predetermined number (e.g., 12) of the preceding most recent measurements to thereby generate the short term running average. Determining the short-term average of the thoracic impedance signal is not limited to the described method. For example, more or less measurements can be made per day, and the short-term average of the thoracic impedance signal can be an average of more or less measurements.

In accordance with an embodiment, the thoracic impedance signal analyzed in the following steps can be a raw thoracic impedance signal (or filtered version thereof). Alternatively, and preferably, the thoracic impedance signal can be a short-term average of measurements obtained from the raw or filtered thoracic impedance signal. At step 204, a duration metric indicative of a duration of drop of the thoracic impedance signal, a magnitude metric indicative of a magnitude of drop of the thoracic impedance signal, and a rate metric indicative of a rate of drop of the thoracic impedance signal is determined based on the obtained thoracic impedance signal. In accordance with an embodiment, the duration metric, magnitude metric, and rate metric determined at step 204 can be determined a predetermined number of times per day (e.g., once per day or every 2 hours), or in response to a triggering event, using the short-term average of the thoracic impedance signal. In accordance with an embodiment, preferably the duration metric, magnitude metric, and rate metric are determined using a short-term average of measurements obtained from the raw thoracic impedance signal. Alternatively, or additionally, in accordance with an embodiment, the metrics determined at step 204 are only determined when scheduled if the slope of the thoracic impedance signal is negative.

In accordance with an embodiment, the duration metric can be determined by taking a first derivative of the thoracic impedance signal, and then determining the length of time the first derivative of the thoracic impedance signal is negative. In accordance with an embodiment, the magnitude metric can be determined by determining the total drop in impedance during the duration that the first derivative of the thoracic impedance signal is negative. In accordance with an embodiment, the rate metric can be determined by dividing the magnitude metric by the duration metric, thus giving the rate of drop of the thoracic impedance signal for a given time. Use of alternative techniques for determining how long the thoracic impedance signal drops can be used to determine the duration metric, while still being within the scope of the present invention. Use of alternative techniques for determining the magnitude of drop of the thoracic impedance signal can be used to determine the magnitude metric, while still being within the scope of the present invention. Use of alternative techniques for determining the rate of drop of the thoracic impedance signal can be used to determine the rate metric, while being within the scope of the present invention.

At step 206, the fluid accumulation level is monitored based on the duration metric, the magnitude metric and the rate metric. For example, the duration metric can be compared to a duration threshold, the magnitude metric can be compared to a magnitude threshold, and the rate metric can be compared to a rate threshold. An abnormal fluid accumulation level can be detected when at least one, two and/or all of the metrics reach their respective threshold. In accordance with an embodiment, a threshold can be reached when a metric equals or exceeds the corresponding threshold. Step 206 can be performed, e.g., once per day, or every time a new impedance measurement is obtained (e.g., every two hours). Other variations are also possible, and within the scope of the present invention.

Additionally, in accordance with an embodiment, the cause of the abnormal fluid accumulation can be determined when at least one, two and/or all of the metrics reach their respective threshold. For example, detecting a rate metric exceeding its rate threshold, and a magnitude metric exceeding its magnitude threshold, can be indicative of abnormal fluid accumulation caused by heart failure, infection or malignancy.

Usefully, a patient's fluid accumulation level can be monitored on a chronic basis because implanted electrodes are used to obtain the thoracic impedance signal. Thus, the patient's fluid accumulation level can be tracked to monitor the patient's evolving cardiac health.

Additionally, an alert, therapy and/or adjusting therapy can be triggered in response to detecting an abnormal fluid level based on the duration metric, the magnitude metric, and the rate metric, as indicated at step 208.

Various different types of alerts may be triggered at step 208. For example, an alert triggering mechanism can be part of an implanted system. Alternatively, an implanted system can trigger a non-implanted alarm of a non-implanted system. In still other embodiments, where fluid accumulation information is transmitted, e.g., via telemetry to an external device, a non-implanted alert can be triggered.

In accordance with an embodiment, therapy can include delivering or instructing the taking of medication, e.g., to restore proper fluid levels, communicating instructions for diet compliance, (e.g., a notice to reduce salt intake), or a reminder for exercise compliance, but is not limited thereto. Adjusting therapy can include, e.g., adjusting drug therapy, which can include alerting a patient to adjust their medication or to visit a physician to get their medication adjusted. Adjusting drug therapy can include adjusting an amount or type of a drug that is being delivered. Alternatively, or additionally, adjusting therapy can include adjusting cardiac resynchronization therapy (CRT), (e.g., adjusting AV delay, VV delay, pacing rate and/or pacing location), and/or communicating with another implanted device (e.g., an insulin or other drug pump, or a neurostimulator) in order to trigger a new or modified therapy.

Referring back to step 206, abnormal fluid accumulatior detection criteria (e.g., the abovementioned thresholds) can be set, and when that detection criteria is/are satisfied, an alert, therapy and/or adjusting therapy is triggered (at step 208). For example, based on a patient's history of detected abnormal fluid accumulation levels, and the accompanying thoracic impedance signal, the duration threshold, the magnitude threshold, and/or the rate threshold can be optimized to detect an abnormal fluid accumulation level. This can include obtaining a thoracic impedance signal during periods of time when the patient is experiencing a normal level of fluid accumulation, and obtaining a thoracic impedance signal during periods of time when the patient transitions from a normal level of fluid accumulation to an abnormal level of fluid accumulation. Based on such information, the duration threshold, the magnitude threshold and the rate threshold can be defined in a manner that reduces the chance of false positives. Additionally, or alternatively, such thresholds can be defined based on thoracic impedance signals obtained from a broad patient population.

An exemplary duration threshold is ten (10) days, meaning the duration threshold is exceeded if the thoracic impedance signal drops for at least ten consecutive days. An exemplary magnitude threshold is 300 ohms, meaning the magnitude threshold is exceeded if the thoracic impedance signal drops at least 300 ohms. An exemplary rate threshold is 30 ohms/day, meaning the rate threshold is exceeded if the thoracic impedance drops at a rate of at least 30 ohms per day. These are just example threshold that are not meant to be limiting.

In accordance with an embodiment, in response to detecting an abnormal fluid accumulation level, at least one of the metrics is compared to a secondary threshold. For example, the duration metric can be compared to a secondary duration threshold, the magnitude metric can be compared to a secondary magnitude threshold, and/or the rate metric can be compared to a secondary rate threshold. Based on the comparison(s) of the selected metric(s) to its secondary threshold, a level of severity of the abnormal fluid accumulation can be determined, as indicated at step 210. For example, detecting a rate metric indicative of a rate of drop of the thoracic impedance signal greater than 40 ohms per day, and a magnitude metric indicative of a magnitude of drop of the thoracic impedance signal greater than 350 ohms, can be indicative of a severe abnormal fluid accumulation level.

In accordance with an embodiment, the duration metric, the magnitude metric, and/or the rate metric can be compared to one or more secondary thresholds to determine a level of severity of an abnormal fluid accumulation level. For example, the duration metric can be compared to a first secondary duration threshold indicative of a moderate fluid accumulation level, and/or a second secondary duration threshold indicative of a severe fluid accumulation level. Based on the comparison(s) of the duration metric to the one or more secondary duration thresholds, a level of severity (e.g., mild, moderate or severe) of the detected abnormal fluid accumulation can be determined. For example, a duration metric that reaches a duration threshold but not a first secondary duration threshold is indicative of a mild abnormal fluid accumulation level, a duration metric that reaches a first secondary duration threshold but not a second secondary duration threshold is indicative of a moderate abnormal fluid accumulation level, and a duration metric that reaches the second secondary duration threshold is indicative of a severe abnormal fluid accumulation level.

In accordance with an embodiment, the result(s) of comparing one or more metrics to one or more secondary thresholds indicative of a level of severity of an abnormal fluid accumulation level can have implications for treatment. For example, if any one severe threshold is reached the patient can be instructed to immediately visit a physician, or if any two moderate thresholds are reached therapy can be modified, or if no moderate thresholds are reached the patient may be instructed to modify their diet and/or increase exercise compliance. Use of alternative threshold crossing schemes and/or treatments associated therewith, can be used while being within the scope of the present invention.

Additionally, in accordance with an embodiment, in response to detecting an abnormal fluid accumulation level, an acceleration metric indicative of an acceleration of drop of the thoracic impedance signal can be determined. In accordance with an embodiment, the acceleration metric can be determined by taking the first derivative of the thoracic impedance signal at two instants of time, determining the difference between the first derivative for the two instants of time, and then dividing the result by the interval of time between the two instants of time. Use of alternative techniques for determining the acceleration of drop of the thoracic impedance signal can be used to determine the acceleration metric, while still being within the scope of the present invention. Additionally, or alternatively, the acceleration metric can be determined by taking the second derivative of the thoracic impedance signal. Use of alternative techniques for determining how fast the rate of drop of the thoracic impedance signal increases can be used to determine the acceleration metric, while still being within the scope of the present invention.

In accordance with an embodiment, the acceleration metric can be determined at step 204 in addition to the duration metric, the magnitude metric, and the rate metric, and compared to its own threshold, with results of this comparison also used to monitor the fluid accumulation level at step 206. In accordance with an embodiment, the thresholds for the duration metric, the magnitude metric, and/or the rate metric can be modified based on the acceleration metric. For example, an acceleration metric that reaches an acceleration threshold can be an indication of a rapidly increasing fluid accumulation level. To account for this, the thresholds for the duration metric, the magnitude metric, and the rate metric can be adjusted (e.g., the thresholds can be lowered) to trigger an earlier alert or therapy to more quickly detect and treat an abnormal fluid accumulation level. It is also possible that the duration threshold, the magnitude threshold, and/or the rate threshold be adjusted according to an equation based on the acceleration metric. For example, the acceleration metric can be in the denominator of an equation such that the higher the acceleration metric the lower the threshold(s) for the other metric(s). For another example, the acceleration metric can be used categorically to select one of several predetermined, discrete threshold levels for the other metric(s).

Based on the acceleration metric, an onset of the detected abnormal fluid accumulation level can be characterized. For example, the acceleration metric can be compared to an acceleration threshold to discriminate between a sudden onset edema and a slow-steady onset edema, which can have implications for different diagnosis, treatment and prognosis. A sudden onset can be precipitated by a cold or infection, while a more gradual onset can be related to a change in diet or activity patterns of the patient. In accordance with an embodiment, appropriate thresholds can be set for characterizing the acceleration metric to determine whether the onset is sudden or gradual. These thresholds can be determined by compiling a database of known acceleration metrics, along with their respective characterization of a fluid accumulation level (e.g., as sudden or gradual). Additionally, the thresholds can be optimized for individual patients based on the patient's history of detected abnormal fluid accumulation levels and the accompanying acceleration metric. In this manner, a general database of known acceleration metrics, along with their respective characterization of a fluid accumulation level, and/or the patient's history of detected abnormal fluid accumulation levels and the accompanying acceleration metric, can be used for determining the appropriate threshold that indicates whether the edema onset is sudden or gradual.

In accordance with an embodiment, the recovery period (the period of time after an alert and/or during/after the therapy), may also be monitored using the acceleration metric, and used to monitor the efficacy of the therapy. For example, the acceleration metric can be indicative of the rate of fluid accumulating (e.g., a negative acceleration metric) in a patient's thoracic cavity, or the rate of fluid dissipation (e.g., a positive acceleration metric) from a patient's thoracic cavity. Thus, a positive secondary acceleration threshold can be used to detect whether a therapy had an immediate effect. Additionally, a negative acceleration secondary threshold can be used to detect whether a therapy had little or no effect, or possibly an adverse effect. More specifically, obtained acceleration metrics can be compared to such secondary acceleration threshold(s) to monitor effects of therapy.

Referring to step 212, information indicative of the patient's monitored fluid accumulation level, and potentially other information, can be stored within memory of the implantable system for later analysis within the device and/or for later transmission to an external device. Such an external device (e.g., an external programmer or external monitor) can then be used to analyze such data.

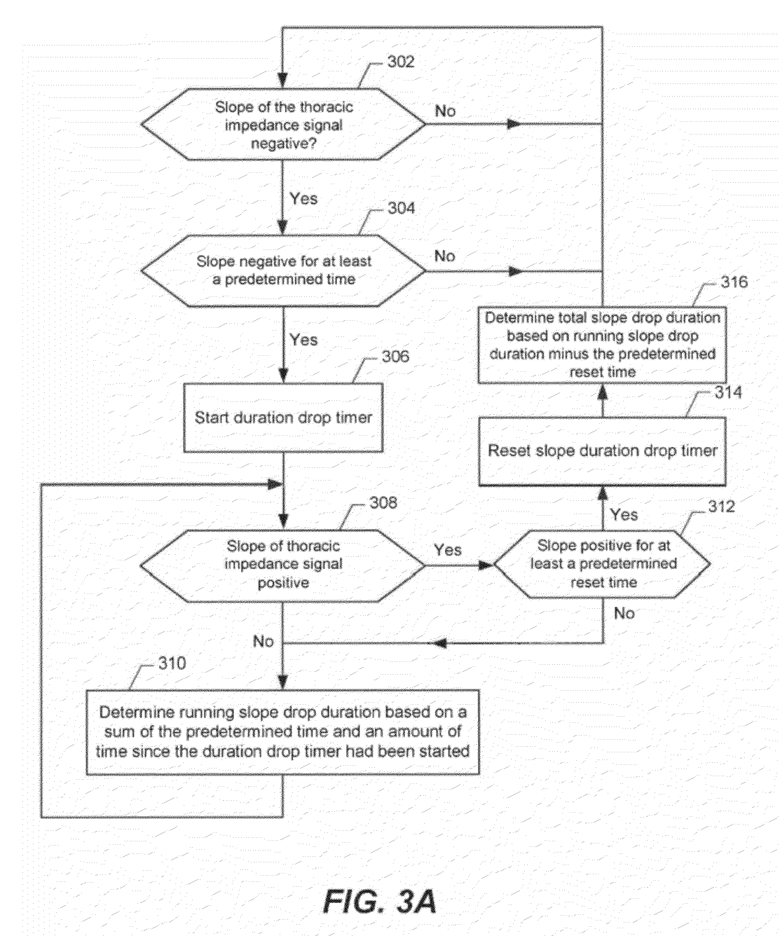

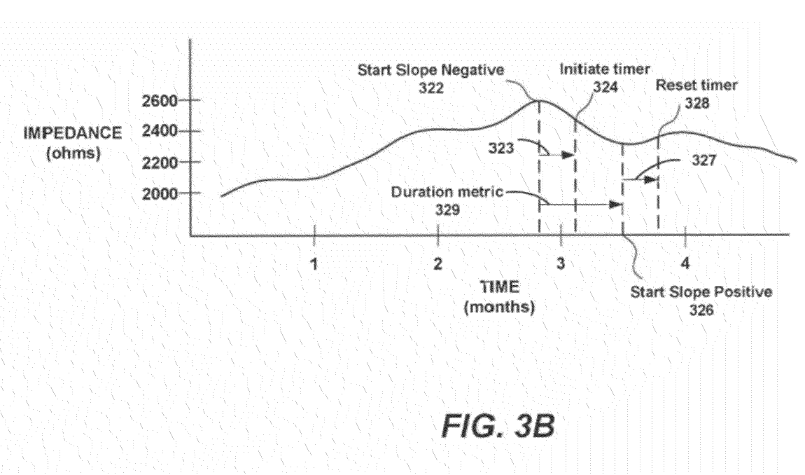

As mentioned above, at step 204 one of the metrics that is determined is a duration metric indicative of a duration of drop of the thoracic impedance signal. FIG. 3A is a high level flow diagram that is used to explain specific embodiments of the present invention that can be used for determining such a duration metric indicative of a duration of drop of the thoracic impedance signal. Referring to FIG. 3A, at decision block 302, it is determined whether the slope of the thoracic impedance signal is negative. If the slope of the signal is negative, then at decision block 304, it is determined whether the slope of the signal has been negative for at least a predetermined time (e.g., the predetermined time can be 48 hours, but is not limited thereto). At step 306, if the slope has been negative for at least the predetermined time, a timer is started to determine the total slope drop duration of the thoracic impedance signal. Alternatively, if it is determined at block 302 that the slope of the thoracic impedance signal is positive, or it is determined at block 304 that the slope of the thoracic impedance signal has been negative for less than the predetermined time, then decision block 302 is repeated to determine whether the slope of the thoracic impedance signal is negative.

At decision block 308, it is determined whether the slope of the thoracic impedance signal changes to positive. If the signal has not changed to positive, then at step 310, a running slope drop duration is determined based on the sum of the predetermined time at step 304 (e.g., 48 hours), and an amount of time since the duration drop timer has been started, e.g., by adding the predetermined time (e.g., 48 hours) and the amount of time since the duration drop timer started. As long as the slope of the thoracic impedance signal is negative, and/or the slope of the signal changes to positive for less than a predetermined reset time (e.g., 1 hour), the running slope drop duration is updated. However, the duration drop timer is reset when the slope of the thoracic impedance signal changes to positive for at least a predetermined reset time (e.g., 1 hour), as demonstrated at decision block 312 and step 314. At step 316, upon resetting the timer, the total slope drop duration is determined based on the running slope drop duration minus the predetermined reset time. The running slope drop duration and/or the total slope drop duration can be the duration metric that is compared to the duration threshold at step 204.

FIG. 3B illustrates an exemplary thoracic impedance signal, and is used to explain how a duration metric indicative of a duration of drop of the thoracic impedance signal can be determined, in accordance with the embodiment described with reference to FIG. 3A. Referring to FIG. 3B, the plot is of impedance (measured in ohms) versus time (measured in months). At time 322, the slope of the thoracic impedance signal is detected as becoming negative. At time 324, which is the time after which the thoracic impedance signal has been negative for a predetermined amount of time 323 (e.g., 48 hours), a timer is started. The timer will continue incrementing until the slope of the signal changes to positive for at least a predetermined reset time (e.g., 1 hour). At time 326, the slope of the thoracic impedance signal is detected as becoming positive. Since at time 328 the slope had remained positive for at least the predetermined reset time 327 (e.g., 1 hour), the timer is reset. Prior to the timer being reset, the duration metric can be the running slope drop duration, which can equal an amount of time since the duration drop timer has been started plus the predetermined amount of time 323 (e.g., 48 hours). Once the timer is reset, the final slope drop duration 329 (which can be used as the duration metric) can be determined as the duration from when the slope became negative (time 322) to when the slope became positive (time 326), after which it remained positive for at least the predetermined reset time (e.g., 1 hour). More specifically, the final slope drop duration can be equal to the value of the timer at the time (328) just prior to the timer being reset, plus the predetermined amount of time (323), minus the predetermined reset time (327). It is noted that in FIG. 3B the various durations shown are not necessarily drawn to scale.

Embodiments of the present invention are not limited to the exact order and/or boundaries of the steps shown in FIGS. 2 and 3A. In fact, many of the steps can be performed in a different order than shown, and many steps can be combined, or separated into multiple steps. For another example, certain steps shown in the FIGS. 2 and 3A can be separated into two or more steps. The only time order that is important is where a step acts on the results of a previous step.

### Exemplary Implantable System

FIGS. 4 and 5 will now be used to describe an exemplary implantable system that can be used to implement embodiments of the present invention including but not limited to monitoring fluid accumulation in a patient's thoracic cavity. Referring to FIG. 4, the implantable system is shown as including an implantable stimulation device 410, which can be a pacing device and/or an implantable cardioverter defibrillator. The device 410 is shown as being in electrical communication with a patient's heart 412 by way of three leads, 420, 424 and 430, which can be suitable for delivering multi-chamber stimulation and shock therapy. The leads can also be used to obtain a thoracic impedance signal signals, for use in embodiments of the present invention.

To sense atrial cardiac signals and to provide right atrial chamber stimulation therapy, the device 410 is coupled to an implantable right atrial lead 420 having at least an atrial tip electrode 422, which typically is implanted in the patient's right atrial appendage. To sense left atrial and ventricular cardiac signals and to provide left-chamber pacing therapy, the device 410 is coupled to a “coronary sinus” lead 424 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 424 is designed to receive atrial and ventricular cardiac signals and to deliver left ventricular pacing therapy using at least a left ventricular tip electrode 426, left atrial pacing therapy using at least a left atrial ring electrode 427, and shocking therapy using at least a left atrial coil electrode 428.

The device 410 is also shown in electrical communication with the patient's heart 412 by way of an implantable right ventricular lead 430 having, in this embodiment, a right ventricular tip electrode 432, a right ventricular ring electrode 434, a right ventricular (RV) coil electrode 436, and an SVC coil electrode 438. Typically, the right ventricular lead 430 is transvenously inserted into the heart 412 so as to place the right ventricular tip electrode 432 in the right ventricular apex so that the RV coil electrode 436 will be positioned in the right ventricle and the SVC coil electrode 438 will be positioned in the superior vena cava. Accordingly, the right ventricular lead 430 is capable of receiving cardiac signals and delivering stimulation in the form of pacing and shock therapy to the right ventricle.

FIG. 5 will now be used to provide some exemplary details of the components of the implantable devices 410. Referring now to FIG. 5, the implantable devices 410, and alternative versions thereof, can include a microcontroller 560. As is well known in the art, the microcontroller 560 typically includes a microprocessor, or equivalent control circuitry, and can further include RAM and/or ROM memory, logic and timing circuitry, state machine circuitry and/or I/O circuitry. Typically, the microcontroller 560 includes the ability to process or monitor input signals (data) as controlled by a program code stored in a designated block of memory. The details of the design of the microcontroller 560 are not critical to the present invention. Rather, any suitable microcontroller 560 can be used to carry out the functions described herein. The use of microprocessor-based control circuits for performing timing and data analysis functions are well known in the art. In specific embodiments of the present invention, the microcontroller 560 performs some or all of the steps associated with monitoring fluid accumulation in a patient's thoracic cavity. Additionally, the microcontroller 560 may detect arrhythmias, and select and control delivery of anti-arrhythmia therapy.

Representative types of control circuitry that may be used with embodiments of the present invention include the microprocessor-based control system of U.S. Pat. No. 4,940,052 (Mann et. al.) and the state-machines of U.S. Pat. No. 4,712,555 (Sholder) and U.S. Pat. No. 4,944,298 (Sholder). For a more detailed description of the various timing intervals used within the pacing device and their inter-relationship, see U.S. Pat. No. 4,788,980 (Mann et. al.). The '052, '555, '298 and '980 patents are incorporated herein by reference.

Depending on implementation, the device 410 can be capable of treating both fast and slow arrhythmias with stimulation therapy, including pacing, cardioversion and defibrillation stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with pacing, cardioversion and defibrillation stimulation. For example, if the implantable device is a monitor that does not provide any therapy, it is clear that many of the blocks shown may be eliminated.

The housing 440, shown schematically in FIG. 5, is often referred to as the “can”, “case” or “case electrode” and may be programmably selected to act as the return electrode for all “unipolar” modes. The housing 440 may further be used as a return electrode alone or in combination with one or more of the coil electrodes, 428, 436 and 438, for shocking purposes. The housing 440 car further include a connector (not shown) having a plurality of terminals, 542, 544, 546, 548, 552, 554, 556, and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522.

To achieve left atrial and ventricular sensing, pacing and shocking, the connector includes at least a left ventricular tip terminal (V_{L} TIP) 544, a left atrial ring terminal (A_{L} RING) 546, and a left atrial shocking terminal (A_{L} COIL) 548, which are adapted for connection to the left ventricular tip electrode 526, the left atrial ring electrode 527, and the left atrial coil electrode 528, respectively.

To support right ventricle sensing, pacing and shocking, the connector further includes a right ventricular tip terminal (V_{R} TIP) 552, a right ventricular ring terminal (V_{R} RING) 554, a right ventricular shocking terminal (R_{V} COIL) 556, and an SVC shocking terminal (SVC COIL) 558, which are adapted for connection to the right ventricular tip electrode 432, right ventricular ring electrode 434, the RV coil electrode 436, and the SVC coil electrode 438, respectively.

An atrial pulse generator 570 and a ventricular pulse generator 572 generate pacing stimulation pulses for delivery by the right atrial lead 420, the right ventricular lead 430, and/or the coronary sinus lead 424 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators, or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry 579 which is used to control pacing parameters (e.g., the timing of stimulation pulses) as well as to keep track of the timing of refractory periods, noise detection windows, evoked response windows, alert intervals, marker channel timing, duration metrics, etc., which is well known in the art. Examples of pacing parameters include, but are not limited to, atrio-ventricular delay, interventricular delay and interatrial delay.

The switch bank 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, etc.) by selectively closing the appropriate combination of switches (not shown) as is known in the art.

Atrial sensing circuits 582 and ventricular sensing circuits 584 may also be selectively coupled to the right atrial lead 420, coronary sinus lead 424, and the right ventricular lead 430, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers, or shared amplifiers. The switch 574 determines the “sensing polarity” of the cardiac signal by selectively closing the appropriate switches, as is also known in the art. In this way, the clinician may program the sensing polarity independent of the stimulation polarity.

Each sensing circuit, 582 and 584, preferably employs one or more low power, precision amplifiers with programmable gain and/or automatic gain control, band-pass filtering, and a threshold detection circuit, as known in the art, to selectively sense the cardiac signal of interest. The automatic gain control enables the device 410 to deal effectively with the difficult problem of sensing the low amplitude signal characteristics of atrial or ventricular fibrillation. Such sensing circuits, 582 and 584, can be used to determine cardiac performance values used in the present invention. Alternatively, an automatic sensitivity control circuit may be used to effectively deal with signals of varying amplitude.

The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity, in the appropriate chambers of the heart. The sensing circuits, 582 and 584, in turn, receive control signals over signal lines, 586 and 588, from the microcontroller 560 for purposes of measuring cardiac performance at appropriate times, and for controlling the gain, threshold, polarization charge removal circuitry (not shown), and timing of any blocking circuitry (not shown) coupled to the inputs of the sensing circuits, 582 and 586.

As further shown in FIG. 5, the device 410 is also shown as having an impedance measuring and processing circuit 513 which is enabled by the microcontroller 560 via a control signal 514 and can be used for obtaining many types of bodily, intracardiac impedances, and thoracic impedances, including a network of single or multi-vector impedance measurements. Such impedance measurements can be used, e.g., for trending many kinds of physiological variables, and can also be used for detection of air movement in and out of the lungs, blockage of airways, lead impedance surveillance during acute and chronic phases for proper lead positioning or dislodgement; lead integrity by detecting insulation abrasion, operable electrodes, and automatically switching to an operable pair if dislodgement occurs; measuring respiration or minute ventilation; measuring thoracic impedance for determining shock thresholds and/or for determining a duration metric indicative of a duration of drop of the thoracic impedance signal, a magnitude metric indicative of a magnitude of drop of the thoracic impedance signal, and a rate metric indicative of a rate of drop of the thoracic impedance signal is determined to monitor a fluid accumulation in the patient; detecting when the device has been implanted; measuring cardiac stroke volume; detecting the opening of heart valves; and so forth. The impedance measuring circuit 513 may be coupled to the switch 574 so that any desired electrodes may be used, and networks of vectors can be selected.

A thoracic impedance signal can be generated substantially continually, periodically, or aperiodically. In an embodiment, a thoracic impedance signal comprises a plurality of discrete thoracic impedance measurements taken at different points in time. In accordance with an embodiment, to generate a thoracic impedance Zₘ measurement, a pacer timing and a control circuit initiates, via microcontroller 560, delivery of a predetermined voltage pulse Vₒ from an output circuit along an excitation path between, for example, the left ventricular tip electrode 426 and the housing 440 of FIG. 4. A resistor Rₒ having a known resistance is incorporated in the output circuit, positioned along the excitation path so that the current delivered along the excitation path can be calculated, using Ohm's Law, as Iₒ=Vₒ/Rₒ. The voltage Vₘ is measured across the measurement path between a point after resistor Rₒ and electrode, and, knowing the current Iₒ delivered to the measurement path, impedance Zₘ is calculated as Zₘ=Vₘ/(Vₒ/Rₒ). This technique for obtaining a thoracic impedance signal is included for exemplary purposes. Other techniques for obtaining a thoracic impedance signal are also within the scope of the present invention.

In accordance with an embodiment, thoracic impedance measurements may, for example, be obtained from pre-programmed vectors, such as the right ventricular tip electrode 432 to the case electrode 440 and/or the atrial tip electrode 422 to the case electrode 440. Referring to FIG. 4, the right ventricular tip electrode 432 and the case electrode 440 may, for example, be utilized for both the excitation path and the measurement path. However, it is understood that other arrangements can also be utilized, such as an arrangement in which the excitation path is between the left ventricular tip electrode 426 and the case electrode 440 and the measurement path is between the left atrial ring electrode 427 and the case electrode 440. It is further contemplated that the leads can be epicardial leads and/or subcutaneous leads.

In accordance with an embodiment of the present invention, the implantable device 410 includes a fluid accumulation monitor 569. The fluid accumulation monitor 569 can be used to monitor the patient's fluid accumulation using the techniques described above with reference to FIGS. 2, 3A and 3B. Such techniques can include determining based on the thoracic impedance signal, a duration metric indicative of a duration of drop of the thoracic impedance signal, a magnitude metric indicative of a magnitude of drop of the thoracic impedance signal, and a rate metric indicative of a rate of drop of the thoracic impedance signal, and then monitoring a fluid accumulation level based on determined metrics. The fluid accumulation monitor 569 can also be configured to monitor changes in the patient's fluid accumulation by monitoring changes in the thoracic impedance signal over time, and can store, within the implantable system, information indicative of the monitored fluid accumulation so that the stored information is available for transfer to a non-implanted system. Based on these changes in the patient's fluid accumulation, fluid accumulation monitor 569 can trigger an alert, therapy and/or adjust therapy.

The fluid accumulation monitor 569 can be implemented within the microcontroller 560, as shown in FIG. 5, and can be implemented by software, firmware, or combinations thereof. It is also possible that all, or portions of the fluid accumulation monitor 569 can be implemented separate from the microcontroller 560.

The implantable device can also include a medication pump 503, which can deliver medication to a patient if the patient's fluid accumulation falls outside certain thresholds or ranges. Information regarding implantable medication pumps may be found in U.S. Pat. No. 4,731,051 (Fischell) and in U.S. Pat. No. 4,947,845 (Davis), both of which are incorporated by reference herein.

For arrhythmia detection, the device 410 includes an arrhythmia detector 562 that utilizes the atrial and ventricular sensing circuits, 582 and 584, to sense cardiac signals to determine whether a rhythm is physiologic or pathologic. The timing intervals between sensed events (e.g., P-waves, R-waves, and depolarization signals associated with fibrillation) can be classified by the microcontroller 560 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to assist with determining the type of remedial therapy that is needed (e.g., bradycardia pacing, anti-tachycardia pacing, cardioversion shocks or defibrillation shocks, collectively referred to as “tiered therapy”). Additionally, the arrhythmia detector 562 can perform arrhythmia discrimination, e.g., using measures of arterial blood pressure determined in accordance with embodiments of the present invention. The arrhythmia detector 562 can be implemented within the microcontroller 560, as shown in FIG. 5. Thus, this detector 562 can be implemented by software, firmware, or combinations thereof. It is also possible that all, or portions, of the arrhythmia detector 562 can be implemented using hardware. Further, it is also possible that all, or portions, of the ischemia detector 562 can be implemented separate from the microcontroller 560.

The implantable device 410 can also include a pacing controller 566, which can adjust a pacing rate and/or pacing intervals based the fluid accumulation level, in accordance with embodiments of the present invention. The pacing controller 566 can be implemented within the microcontroller 560, as shown in FIG. 5. Thus, the pacing controller 566 can be implemented by software, firmware, or combinations thereof. It is also possible that all, or portions, of the pacing controller 566 can be implemented using hardware. Further, it is also possible that all, or portions, of the pacing controller 566 can be implemented separate from the microcontroller 560.

Still referring to FIG. 5, cardiac signals are also applied to the inputs of an analog-to-digital (A/D) data acquisition system 590. The data acquisition system 590 can be configured to acquire various signals, including but not limited to, CI, IEGM, PPG and IPG signals, convert the raw analog data into a digital signal, and store the digital signals for later processing and/or telemetric transmission to an external device 502. The data acquisition system 590 can be coupled to the right atrial lead 420, the coronary sinus lead 424, and the right ventricular lead 430 through the switch 574 to sample cardiac signals across any pair of desired electrodes.

The data acquisition system 590 can be coupled to the microcontroller 560, or other detection circuitry, for detecting an evoked response from the heart 412 in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 560 detects a depolarization signal during a window following a stimulation pulse, the presence of which indicates that capture has occurred. The microcontroller 560 enables capture detection by triggering the ventricular pulse generator 572 to generate a stimulation pulse, starting a capture detection window using the timing control circuitry 579 within the microcontroller 560, and enabling the data acquisition system 590 via control signal 592 to sample the cardiac signal that falls in the capture detection window and, based on the amplitude, determines if capture has occurred.

The implementation of capture detection circuitry and algorithms are well known. See for example, U.S. Pat. No. 4,729,376 (Decote, Jr.); U.S. Pat. No. 4,708,142 (Decote, Jr.); U.S. Pat. No. 4,686,988 (Sholder); U.S. Pat. No. 4,969,467 (Callaghan et. al.); and U.S. Pat. No. 5,350,410 (Mann et. al.), which patents are hereby incorporated herein by reference. The type of capture detection system used is not critical to the present invention.

The microcontroller 560 is further coupled to the memory 594 by a suitable data/address bus 596, wherein the programmable operating parameters used by the microcontroller 560 are stored and modified, as required, in order to customize the operation of the implantable device 410 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart 412 within each respective tier of therapy. The memory 594 can also store data including information about the patient's fluid accumulation level.

The operating parameters of the implantable device 410 may be non-invasively programmed into the memory 594 through a telemetry circuit 501 in telemetric communication with an external device 502, such as a programmer, transtelephonic transceiver, or a diagnostic system analyzer. The telemetry circuit 501 can be activated by the microcontroller 560 by a control signal 506. The telemetry circuit 501 advantageously allows intracardiac electrograms and status information relating to the operation of the device 410 (as contained in the microcontroller 560 or memory 594) to be sent to the external device 502 through an established communication link 505. The telemetry circuit can also be use to transmit arterial blood pressure data to the external device 502.

For examples of telemetry devices, see U.S. Pat. No. 4,809,697, entitled “Interactive Programming and Diagnostic System for use with Implantable Pacemaker” (Causey, III et al.); U.S. Pat. No. 4,944,299, entitled “High Speed Digital Telemetry System for Implantable Device” (Silvian); and U.S. Pat. No. 6,275,734 entitled “Efficient Generation of Sensing Signals in an Implantable Medical Device such as a Pacemaker or ICD” (McClure et al.), which patents are hereby incorporated herein by reference.

The implantable device 410 additionally includes a battery 511 which provides operating power to all of the circuits shown in FIG. 5. If the implantable device 410 also employs shocking therapy, the battery 511 should be capable of operating at low current drains for long periods of time, and then be capable of providing high-current pulses (for capacitor charging) when the patient requires a shock pulse. The battery 511 should also have a predictable discharge characteristic so that elective replacement time can be detected.

The implantable device 410 is also shown as including an activity and/or posture sensor 515. Such a sensor 515 can be a simple one dimensional sensor that converts mechanical motion into a detectable electrical signal, such as a back electro magnetic field (BEMF) current or voltage, without requiring any external excitation. Alternatively, the sensor 515 can measure multi-dimensional activity information, such as two or more of acceleration, direction, posture and/or tilt. Examples of multi-dimensional activity sensors include, but are not limited to: the three dimensional accelerometer-based position sensor disclosed in U.S. Pat. No. 6,658,292 to Kroll et al., which is incorporated herein by reference; the AC/DC multi-axis accelerometer disclosed in U.S. Pat. No. 6,466,821 to Pianca et al., which in incorporated herein by reference; and the commercially available precision dual-axis accelerometer model ADXL203 and three-axis accelerometer model ADXL346, both available from Analog Devices of Norwood, Mass.

The implantable device 410 can also include a magnet detection circuitry (not shown), coupled to the microcontroller 560. It is the purpose of the magnet detection circuitry to detect when a magnet is placed over the implantable device 410, which magnet may be used by a clinician to perform various test functions of the implantable device 410 and/or to signal the microcontroller 560 that the external programmer 502 is in place to receive or transmit data to the microcontroller 560 through the telemetry circuits 501.

In the case where the implantable device 410 is also intended to operate as an implantable cardioverter/defibrillator (ICD) device, it should detect the occurrence of an arrhythmia, and automatically apply an appropriate electrical shock therapy to the heart aimed at terminating the detected arrhythmia. To this end, the microcontroller 560 further controls a shocking circuit 516 by way of a control signal 518. The shocking circuit 516 generates shocking pulses of low (up to 0.5 Joules), moderate (0.5-10 Joules), or high energy (11 to 40 Joules), as controlled by the microcontroller 560. Such shocking pulses are applied to the patient's heart 412 through at least two shocking electrodes, and as shown in this embodiment, selected from the left atrial coil electrode 428, the RV coil electrode 436, and/or the SVC coil electrode 438. As noted above, the housing 440 may act as an active electrode in combination with the RV coil 436, or as part of a split electrical vector using the SVC coil electrode 438 or the left atrial coil electrode 428 (i.e., using the RV electrode as a common electrode).

The above described implantable device 410 was described as an exemplary pacing device. One or ordinary skill in the art would understand that embodiments of the present invention can be used with alternative types of implantable devices. Accordingly, embodiments of the present invention should not be limited to use only with the above described device.

Embodiments of the present invention have been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in FIGS. 2 and 3A. Further, it is possible to change the order of some of the steps shown in FIGS. 2 and 3A, without substantially changing the overall events and results.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. For use with an implantable system, a method for monitoring fluid accumulation in a patient's thoracic cavity, the method comprising:
(a) obtaining a thoracic impedance signal for the patient;
(b) determining, based on the thoracic impedance signal,
(b.1) a duration metric indicative of a duration of drop of the thoracic impedance signal,
(b.2) a magnitude metric indicative of a magnitude of drop of the thoracic impedance signal, and
(b.3) a rate metric indicative of a rate of drop of the thoracic impedance signal; and
(c) monitoring a fluid accumulation level based on the duration metric, the magnitude metric and the rate metric.

2. The method of claim 1, further comprises:
(d) triggering an alert, triggering therapy and/or adjusting therapy in response to detecting an abnormal fluid accumulation level.

3. The method of claim 1, wherein
step (b) further comprises determining, based on the thoracic impedance signal, an acceleration metric indicative of an acceleration of drop of the thoracic impedance signal; and
the monitoring the fluid accumulation level at step (c) is also based on the acceleration metric.

4. The method of claim 1, wherein step (c) comprises:
(c.1) comparing the duration metric to a duration threshold;
(c.2) comparing the magnitude metric to a magnitude threshold;
(c.3) comparing the rate metric to a rate threshold; and
(c.4) monitoring an abnormal fluid accumulation level based on results of steps (c.1), (c.2) and (c.3).

5. The method of claim 4, further comprises:
determining, based on the thoracic impedance signal, an acceleration metric indicative of an acceleration of drop of the thoracic impedance signal; and
modifying at least one of the duration threshold, the magnitude threshold, and the rate threshold based on the acceleration metric.

6. The method of claim 4, wherein step (c.4) includes detecting an abnormal fluid accumulation level when at least one of the following events occur:
the duration metric reaches the duration threshold;
the magnitude metric reaches the magnitude threshold; and
the rate metric reaches the rate threshold.

7. The method of claim 4, wherein step (c.4) includes detecting an abnormal fluid accumulation level when at least two of the following events occur:
the duration metric reaches the duration threshold;
the magnitude metric reaches the magnitude threshold; and
the rate metric reaches the rate threshold.

8. The method of claim 4, wherein step (c.4) includes detecting an abnormal fluid accumulation level when all three of the following events occur:
the duration metric reaches the duration threshold;
the magnitude metric reaches the magnitude threshold; and
the rate metric reaches the rate threshold.

9. The method of claim 4, wherein step (c) further comprises:
(c.5) in response to detecting an abnormal fluid accumulation level, performing at least one of the following
(c.5.a) comparing the duration metric to one or more secondary duration threshold;
(c.5.b) comparing the magnitude metric to one or more secondary magnitude threshold; and
(c.5.c) comparing the rate metric to one or more secondary rate threshold; and
(c.6) determining a severity of the abnormal fluid accumulation based on results of step (c.5).

10. The method of claim 8, further comprises:
(d) triggering an alert, triggering therapy and/or adjusting therapy in response to detecting an abnormal fluid accumulation level and based on the determined severity of the fluid accumulation.

11. The method of claim 4, wherein step (c) further comprises:
(c.5) in response to detecting an abnormal fluid accumulation level, determining an acceleration metric indicative of an acceleration of drop of the thoracic impedance signal; and
(c.6) characterizing an onset of the detected abnormal fluid accumulation level based on results of step (c.5).

12. The method of claim 1, further comprising:
(d) storing within the implantable system information indicative of the monitored fluid accumulation level so that the stored information is available for transfer to a non-implanted system.

13. The method of claim 1, wherein step (b.1) comprises using a timer to determine the duration metric, which includes:
starting the timer in response to detecting that a slope of the thoracic impedance signal has been negative for at least a predetermined time;
resetting the timer when the slope of the thoracic impedance signal changes to positive for at least a predetermined reset time;
not resetting the timer when the slope of the thoracic impedance signal changes to positive for less than the predetermined reset time; and
after the slope drop duration time has been started, determining the duration metric based on a sum of the predetermined time and an amount of time since the timer had been started.

14. An implantable system configured to monitor fluid accumulation in a patient's thoracic cavity, comprising:
one or more electrodes configured to obtain a thoracic impedance signal for the patient;
a fluid accumulation monitor configured to determine, based on the thoracic impedance signal,
a duration metric indicative of a duration of drop of the thoracic impedance signal,
a magnitude metric indicative of a magnitude of drop of the thoracic impedance signal, and
a rate metric indicative of a rate of drop of the thoracic impedance signal; and
wherein the fluid accumulation monitor is configured to monitor a fluid accumulation level based on the duration metric, the magnitude metric and the rate metric.

15. The implantable system of claim 14, wherein the fluid accumulation monitor is also configured to trigger an alert, therapy and/or adjust therapy in response to detecting an abnormal fluid accumulation level.

16. The implantable system of claim 14, wherein:
the fluid accumulation monitor is also configured to determine, based on the thoracic impedance signal, an acceleration metric indicative of an acceleration of drop of the thoracic impedance signal, and
the fluid accumulation monitor is configured to monitor the fluid accumulation level also based on the acceleration metric.

17. The implantable system of claim 14, wherein the fluid accumulation monitor is configured to:
compare the duration metric to a duration threshold;
compare the magnitude metric to a magnitude threshold;
compare the rate metric to a rate threshold; and
monitor for the abnormal fluid accumulation level based on results of the comparisons.

18. The implantable system of claim 17, wherein:
the fluid accumulation monitor is also configured to determine, based on the thoracic impedance signal, an acceleration metric indicative of an acceleration of drop of the thoracic impedance signal; and
the fluid accumulation monitor is configured to modify at least one of the duration threshold, the magnitude threshold, and the rate threshold based on the acceleration metric.

19. The implantable system of claim 17, wherein the fluid accumulation monitor is also configured to determine a severity of a detected abnormal fluid accumulation level based on results of comparing at least one of the duration metric, the magnitude metric, and the rate metric to one or more corresponding secondary threshold.

20. The implantable system of claim 17, wherein the fluid accumulation monitor is also configured to characterize an onset of a detected abnormal fluid accumulation level by determining an acceleration metric indicative of an acceleration of drop of the thoracic impedance signal, and comparing the acceleration metric to one or more acceleration threshold.

21. The implantable system of claim 14, further including a timing control mechanism including a timer to determine the duration metric, wherein the timing control mechanism:
starts the timer in response to detecting that a slope of the thoracic impedance signal has been negative for at least a predetermined time;
resets the timer when the slope of the thoracic impedance signal changes to positive for at least a predetermined reset time;
does not reset the timer when the slope of the thoracic impedance signal changes to positive for less than the predetermined reset time; and
after the slope drop duration time has been started, determines the duration metric based on a sum of the predetermined time and an amount of time since the timer had been started.

22. For use with an implantable system, a method comprising:
(a) obtaining a thoracic impedance signal indicative of fluid accumulation within a patient's thoracic cavity;
(b) monitoring, based on the thoracic impedance signal,
a duration of drop of the thoracic impedance signal,
a magnitude of drop of the thoracic impedance signal, and
a rate of drop of the thoracic impedance signal; and
(c) monitoring for an abnormal fluid accumulation level based on the duration of drop, the magnitude of drop and the rate of drop of the thoracic impedance signal.

23. The method of claim 22, further comprising:
(d) in response to detecting an abnormal fluid accumulation level, determining a cause of the abnormal fluid accumulation level based on one or more of the duration of drop, the magnitude of drop and the rate of drop of the thoracic impedance signal.

24. The method of claim 22, further comprising:
(d) in response to detecting an abnormal fluid accumulation level, determining an acceleration of drop of the thoracic impedance signal, and characterizing an onset of the detected abnormal fluid accumulation level based on the acceleration of drop of the thoracic impedance signal.
